(19) United States
(12) Patent Application Publication
(10) Pub. No.: US 2023/0190621 A1
(43) Pub. Date: Jun. 22, 2023

(54) WATER-BASED NAIL POLISH

(71) Applicants: Ashley Ann Thomas, Orange, NJ (US); Devaki Rani Sakhamuru, Euclid, OH (US)

(72) Inventors: Ashley Ann Thomas, Orange, NJ (US); Devaki Rani Sakhamuru, Euclid, OH (US)

(21) Appl. No.: 17/555,330

(22) Filed: Dec. 17, 2021

(51) Int. Cl. A61K 8/73; A61K 8/891; A61K 8/87; A61K 8/81; A61K 8/34; A61K 8/86; A61K 8/41; A61Q 3/02

(52) U.S. Cl. CPC A61K 8/73; A61K 8/891; A61K 8/87; A61K 8/8147; A61K 8/34; A61K 8/86; A61K 8/41; A61K 8/345; A61Q 3/02; A61K 2800/43; A61K 2800/48; A61K 2800/95; A61K 2800/524

(57) ABSTRACT

This invention is directed to a water-based nail polish that provides the desired characteristics for a nail polish and incorporates ingredients that are non-toxic to human and environmentally friendly, fume free, devoid of typical unpleasant odor, incorporated with pleasant scent, and generally fire-resistant. The nail polish includes solvent comprising deionized water, fragrance solution, thickener comprising xanthan gum, film former comprising polyurethane, stabilizer comprising silicone emulsion, drying accelerator comprising acrylates copolymer, and color pigment.

### BACKGROUND

Conventional nail polish in the market today often contains chemicals that are deemed toxic. These toxic chemicals and fumes include toluene, dibutyl phthalate (DBP), formaldehyde resin, camphor, ethyl tosylamide, and xylene, and the like. Many conventional nail polish products also contain nitrocellulose which is considered hazardous waste and generally bad for the environment. These toxic, unpleasant, toxic fumed and environment-unfriendly chemicals are typically used to achieve desired characteristics for a nail polish, which include quick drying, good adhesion, glossy look, and wear resistant. Conventional polish also presents fire hazard in addition to toxic fumes.

### SUMMARY

This invention is directed to a water-based nail polish that provides the desired characteristics for a nail polish and incorporates ingredients that are non-toxic to humans and environmentally friendly, fume free, devoid of typical unpleasant odor, incorporated with pleasant scent, and generally fire-resistant. The nail polish includes solvent comprising deionized water, fragrance solution, thickener comprising xanthan gum, film former comprising polyurethane, stabilizer comprising silicone emulsion, drying accelerator comprising acrylates copolymer, and color pigment.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, and/or components have been described at a relatively high-level, without detail, in order to avoid unnecessarily obscuring aspects of the present teachings.

This invention is directed to a water-based nail polish that provides the desired characteristics for a nail polish along with the novelty of a fume free, pleasant-scented polish and incorporates ingredients that are non-toxic to humans and environmentally friendly. The pleasant-scented nature of the water-based polish enhances the experience and desired characteristics and sets a novel precedent for the nail polish users to allow them to polish anywhere without the off-putting unpleasant odor. In one embodiment, the water-based nail polish includes solvent comprising deionized water, thickener comprising xanthan gum, film former comprising polyurethane, stabilizer comprising silicone emulsion, drying accelerator comprising acrylates copolymer, leveling agent comprising polyurethane rheology modifier, antibacterial and/or antifungal oil, fragrance solution, and color pigment.

An example composition of the water-based nail polish includes the following materials in parts by weight:

15 to 25 parts of deionized water;

0.01 to 1 part of a fragrance solution;

0.01 to 1 part of xanthan gum;

1 to 2 parts of acrylates copolymer;

15 to 20 parts of acrylates copolymer diluted with 40-70% of water;

10 to 15 parts of silicone emulsion;

20 to 25 parts of polyurethane; and

5 to 10 parts of color pigment

The example composition of the water-based nail polish may also include 0.01 to 1 part of antibacterial and/or antifungal oil. The antibacterial and/or antifungal oil may be neem oil, tea tree oil and other oils with antibacterial and/or antifungal properties. Distilled water can be used as deionized water. The fragrance solution in the water-based nail polish is intended to transform and enhance the experience of the manicure during and after the application process. Example fragrance solutions include lavender and eucalyptus. The example composition of the water-based nail polish may further include one or more of the following additional materials in parts by weight:

1 to 5 parts of polyethylene glycol

0.01 to 1 part of phenoxyethanol

0.01 to 1 part of triethanolamine

2 to 5 parts of ethanol

0.01 to 1 part of acrylic copolymer ammonium salt

0.01 to 1 part of alcohol amine

0.01 to 1 part of polyoxyethylene alkyl ether

0.01 to 1 part of polypropionate salt copolymer

0.01 to 1 part of polyurethane rheology modifier

2 to 5 parts of pentanediol

In another embodiment, a water-based nail polish composition includes the ingredients shown in Table 1 in relative percentage by weight:

TABLE 1
Relative Parts & Function of Ingredients

| | | Relative | |
|---|---|---|---|
| Ingredients | Function | Weights | Phase |
| Deionized water | Solvent | 10-15% | A |
| Xanthan Gum | Thickener | 0.01-1% | A |
| Acrylate copolymer | Thickener/Binder | 1-2% | B |
| Ethanol | Solvent | 2-5% | B |
| Triethanolamine | pH Neutralizer | 0.01-1% | B |
| Silicone Emulsion | Film former/Longevity | 10-15% | C |
| Acrylate copolymer | Film Former/Fast | 15-20% | C |
| diluted with 40-70% | drying | | |
| water | | | |
| Deionized water | Solvent | 5-10% | B |
| Pentanediol | Plasticizer | 2-5% | D |
| Antibacterial/Antifungal | Antibacterial/antifungal | 0.01-1% | D |
| oil (such as neem oil) | | | |
| Propylene Glycol | Moisturizer | 2-5% | D |
| Polyurethane | Film Former/Fast | 20-25% | E |
| | drying/Adhesion | | |
| Acrylic Copolymer | Water resistant | 0.01-1% | F |
| Ammonium Salt | | | |
| Polyoxyethylene | Rheology | 0.01-1% | F |
| Alkyl Ether | | | |
| Polyurethane | Leveling Agents | 0.01-1% | F |
| Rheology Modifier | | | |
| Polypropionate Salt | Dispersion | 0.01-1% | F |
| copolymer | | | |
| Alcohol Amine | Scratch/smooth/water | 0.01-1% | F |
| | resistance | | |
| Phenoxy ethenol | Shelflife | 0.01-1% | G |
| Fragrance | Scent | 0.01-1% | G |
| Color pigment | Colorant | 5-10% | H |

An example preparation process for the water-based nail polish composition with ingredients shown in Table 1 includes the following steps:
Measure out and combine phase A by pouring the xanthan gum into the deionized water slowly, just set aside and allow the xanthan gum to be hydrated for at least 5 minutes, then mix until it forms homogeneous solution.
Measure out and mix phase B at low shear until an emulsion/gel is formed.
Combine phase A and phase C using low shear mixing.
Combine phase D with phase B using low shear mixing.
Combine phase AC with phase BD. Add phase E then mix very well.
Add phase F then phase G and H. Mix very well until color pigment disperses.

An example application process for the water-based nail polish composition with ingredients shown in Table 1 includes the following steps:

Before applying, nails must be clear of all residues so use nail polish remover before applying.

Avoid applying any lotion or oils to the nails beforehand.

Apply 1-3 coats of polish.

Apply the water-based topcoat.

Let the polish dry for 5-10 minutes.

The embodiments of the water-based nail polish described herein provides a non-toxic alternative to existing nail polish products in the market. This water-based nail polish does not contain harmful chemicals, such as toluene, dibutyl phthalate (DBP), formaldehyde, formaldehyde resin, camphor, ethyl tosylamide, and xylene. This water-based nail polish also does not contain nitrocellulose which is found in many polishes in the market and may be considered hazardous waste. Instead, the major ingredients of this polish generally do not cause harm to the environment and the users when used as directed.

The inventors found that this polish can last between 3-5 days on the nail. The inventors also found that the acrylate copolymer gives the nail polish fast drying properties. In testing, this nail polish can dry in around 5 minutes, compared to the average of 15-20 minutes. This nail polish does not contain any volatile liquids. As a result, it may be considered safe to store in large quantities and can travel via air. This nail polish may contain relaxing fragrances like lavender and eucalyptus which enhance the manicure experience.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various examples for the purpose of streamlining the disclosure. This method of disclosure is not to be interpreted as reflecting an intention that the claims require more features than are expressly recited in each claim. Rather, as the following claims reflect, inventive subject matter lies in less than all features of a single disclosed example. Thus, the following claims are hereby incorporated into the Detailed Description, with each claim standing on its own as a separately claimed subject matter.

The embodiments of this water-based nail polish described herein provides a fume free alternative and pleasant scented alternative to existing nail polish products on the market. This nail polish does not contain toxic fumes resulting from nitrocellulose and solvents. Instead the major ingredients of this polish are pleasant scented and not off putting to the user or people around the user who may find the existing nail polish smell to be off putting.

What is claimed is:

1. A nail polish composition comprising the following materials in parts by weight:
15 to 25 parts of deionized water;
0.01 to 1 part of a fragrance solution;
0.01 to 1 part of xanthan gum;
1 to 2 parts of acrylates copolymer;
15 to 20 parts of acrylates copolymer diluted with water;
10 to 15 parts of silicone emulsion;
20 to 25 parts of polyurethane; and
5 to 10 parts of color pigment

2. The nail polish composition of claim 1, further comprising 0.01 to 1 part of antibacterial and/or antifungal oil.

3. The nail polish composition of claim 1, further comprising the following material in parts by weight: 1 to 5 parts of polyethylene glycol.

4. The nail polish composition of claim 1, further comprising the following material in parts by weight: 0.01 to 1 part of phenoxyethanol.

5. The nail polish composition of claim 1, further comprising the following material in parts by weight: 0.01 to 1 part of triethanolamine.

6. The nail polish composition of claim 1, further comprising the following material in parts by weight: 2 to 5 parts of ethanol.

7. The nail polish composition of claim 1, further comprising at least one of the following materials in parts by weight: 2 to 5 parts of pentanediol and 0.01 to 1 part of polyurethane rheology modifier.

8. The nail polish composition of claim 1, further comprising the following material in parts by weight: 0.01 to 1 part of acrylic copolymer ammonium salt.

9. The nail polish composition of claim 1, further comprising the following material in parts by weight: 0.01 to 1 part of alcohol amine.

10. The nail polish composition of claim 1, further comprising the following material in parts by weight: 0.01 to 1 part of polyoxyethylene alkyl ether.

11. The nail polish composition of claim 1, further comprising the following material in parts by weight: 0.01 to 1 part of polypropionate salt copolymer.

12. A preparation method of a water-based nail polish composition comprising:
measuring the following ingredients by relative % by weights:

| | Relative | |
|---|---|---|
| | % by | |
| Ingredients | Weights | Phase |
| Deionized water | 10-15% | A |
| Xanthan gum | 0.01-1% | A |
| Acrylate copolymer | 1-2% | B |
| Ethanol | 2-5% | B |
| Triethanolamine | 0.01-1% | B |
| Silicone emulsion | 10-15% | C |
| Acrylate copolymer diluted | 15-20% | C |
| with 40-70% of water | | |
| Deionized water | 5-10% | B |
| Pentanediol | 2-5% | D |
| Neem oil | 0.01-1% | D |
| Propylene glycol | 2-5% | D |
| Polyurethane | 20-25% | E |
| Acrylic copolymer ammonium | 0.01-1% | F |
| salt | | |
| Polyoxyethylene | 0.01-1% | F |
| alkyl ether | | |
| Polyurethane | 0.01-1% | F |
| rheology modifier | | |
| Polypropionate salt copolymer | 0.01-1% | F |
| Alcohol amine | 0.01-1% | F |
| Phenoxy ethenol | 0.01-1% | G |
| Fragrance | 0.01-1% | G |
| Color pigment | 5-10% | H |

combining a phase A mixture by slowly pouring the xanthan gum into the phase A deionized water;
allowing the xanthan gum in the phase A mixture to be hydrated for at least 5 minutes;
mixing the phase A mixture until a homogeneous solution is formed;
combining phase B ingredients and mixing at low shear until an emulsion or gel is formed to create a phase B mixture;
combining the phase A mixture and phase C ingredients using low shear mixing to form a phase AC mixture;
combining phase D ingredients with the phase B mixture using low shear mixing to form a phase BD mixture;
combining the phase AC mixture with the phase BD mixture to form a phase ABCD mixture;
adding phase E ingredients to the phase ABCD mixture to form a phase ABCDE mixture; and
adding phase F ingredients and then phase G ingredients and phase H ingredients, and mixing until the color pigment disperses to form the water-based nail polish composition.

13. A non-toxic, fume free nail polish comprising the following components:
solvent comprising deionized water;
fragrance solution;
thickener comprising xanthan gum;
film former comprising polyurethane;
stabilizer comprising silicone emulsion;
binder comprising acrylates copolymer;
drying accelerator comprising acrylates copolymer diluted with water; and
color pigment.

14. The non-toxic fume free nail polish of claim 13, further comprising the following component: pH neutralizer comprising triethanolamine.

15. The non-toxic fume free nail polish of claim 13, wherein the solvent further comprising ethanol.

16. The non-toxic fume free nail polish of claim 13, further comprising at least of the following components: preservative comprising phenoxyethanol and leveling agent comprising polyurethane rheology modifier.

17. The non-toxic fume free nail polish of claim 13, wherein the solvent further comprising antibacterial/antifungal oil.

18. The non-toxic fume free nail polish of claim 13, further comprising the following components: water resistant agent comprising acrylic copolymer ammonium salt.

19. The fume free non-toxic nail polish of claim 13, further comprising the following components: pH balancer comprising triethanolamine.

20. The fume free non-toxic nail polish of claim 13, further comprising the following components: scratch agent comprising alcohol amine.
